(19) United States
(12) Patent Application Publication
(10) Pub. No.: US 2020/0008690 A1
(43) Pub. Date: Jan. 9, 2020

(54) BLOOD PRESSURE DATA PROCESSING APPARATUS, BLOOD PRESSURE DATA PROCESSING METHOD, AND PROGRAM

(71) Applicant: OMRON HEALTHCARE CO., LTD., Muko-shi (JP)

(72) Inventors: Ayako KOKUBO, Kyoto (JP); Hirotaka WADA, Kyoto (JP); Hiroshi NAKAJIMA, Kyoto (JP)

(73) Assignee: OMRON HEALTHCARE CO., LTD., Muko-shi (JP)

(21) Appl. No.: 16/561,347

(22) Filed: Sep. 5, 2019

Related U.S. Application Data
(63) Continuation of application No. PCT/JP2018/009583, filed on Mar. 12, 2018.

(30) Foreign Application Priority Data
Mar. 14, 2017 (JP) 2017-048946

(51) Int. Cl. A61B 5/021; A61B 5/00; A61B 5/08

(52) U.S. Cl. CPC A61B 5/02108; A61B 5/7203; A61B 5/0826; A61B 5/7445; A61B 5/02141; A61B 5/7271; A61B 5/6824

(57) ABSTRACT

A blood pressure surge is detected from time-series data regarding a blood pressure value. An acquisition unit configured to acquire time-series data regarding a blood pressure value; a calculation unit configured to set one or more peak detection intervals to the time-series data, and calculate a feature amount for each of the peak detection intervals based on one of a systolic blood pressure, a diastolic blood pressure, and a pulse pressure; and a specifying unit configured to specify at least one first peak from the feature amount of each peak detection interval are included.

### TECHNICAL FIELD

The present invention relates to a technique for processing blood pressure data acquired by a blood pressure measurement apparatus that measures the blood pressure of a subject.

### BACKGROUND ART

It is known that in a patient affected by sleep apnea syndrome (SAS), when breathing is resumed after apnea, the blood pressure suddenly rises and thereafter falls. Hereinafter, this sudden blood pressure fluctuation will be called a “blood pressure surge” (or simply a “surge”). It is thought that blood pressure information relating to surges that occur in a patient (e.g., information regarding the number of instances in which blood pressure surges occur per unit time, and statistics such as the amount of blood pressure fluctuation) is useful in diagnosing and treating SAS.

24-hour ambulatory blood pressure monitoring (ABPM) is blood pressure monitoring that is carried out over twenty-four hours, through which the blood pressure value is measured at several points in time every hour. With such ambulatory blood pressure monitoring, it is impossible to observe a blood pressure fluctuation that occurs in a short period of time, and it is also difficult to detect a surge, which is a sudden blood pressure fluctuation.

JP 2007-282668A discloses integrating pieces of blood pressure value data measured at a plurality of dates using a conventional blood pressure measurement apparatus in order to observe fluctuation of the blood pressure measurement value within a day or a week.

JP 2012-239807A discloses evaluating the risk of cardiovascular disease occurring in a subject, based on a relationship between the blood pressure and the blood oxygen saturation level measured under a hypoxic condition, and calculating a difference between the blood pressure measured under a hypoxic condition and the blood pressure measured under a non-hypoxic condition (the amount of an increase in the blood pressure).

### SUMMARY OF INVENTION

However, a technique for detecting surges from blood pressure value data acquired using a blood pressure measurement apparatus has not been established. Therefore, manual work needs to be carried out by a doctor or the like to acquire blood pressure information related to surges. An enormous amount of time-series data can be acquired regarding the blood pressure value of a sleeping patient. For example, if sleep time per night is eight hours, time-series data can be acquired regarding the blood pressure value corresponding to approximately thirty thousand beats. It is difficult to manually find surges from such blood pressure data.

The present invention has been made with attention given to the above-described circumstances, and an objective of the present invention is to provide a blood pressure data processing apparatus, a blood pressure data processing method, and a program, according to which it is possible to detect blood pressure surges from time-series data regarding the blood pressure value.

To achieve the above objective, the following aspects are employed for the present invention.

In a first aspect, a blood pressure data processing apparatus includes: an acquisition unit configured to acquire time-series data regarding a blood pressure value; a calculation unit configured to set one or more peak detection intervals to the time-series data, and calculate a feature amount for each of the peak detection intervals based on one of a systolic blood pressure, a diastolic blood pressure, and a pulse pressure; and a specifying unit configured to specify at least one first peak from the feature amount of each peak detection interval.

According to the first aspect, it is possible to specify a first peak from a feature amount that is based on one of a systolic blood pressure, a diastolic blood pressure, and a pulse pressure in each peak detection interval of time-series data regarding the blood pressure value. Therefore, it is possible to detect a blood pressure surge as a first peak. If the time-series data is data regarding the blood pressure value at each beat, it is possible to accurately detect blood pressure surges. Also, it is possible to robustly detect blood pressure surges that do not periodically occur and blood pressure surges that have various patterns.

In a second aspect, the feature amount may be a maximum value of one of the systolic blood pressure, the diastolic blood pressure, and the pulse pressure.

In a third aspect, the feature amount may be a difference between the maximum value in the peak detection interval and a minimum value of one of the systolic blood pressure, the diastolic blood pressure, and the pulse pressure at a point in time that precedes the maximum value in the peak detection interval. According to the third aspect, it is possible to detect blood pressure surges where the blood pressure value sharply increases, based on the amount of fluctuation of one of the systolic blood pressure, the diastolic blood pressure, and the pulse pressure in the peak detection interval.

In a fourth aspect, the blood pressure data processing apparatus may further include an extraction unit configured to extract a peak candidate from each peak detection interval by applying a determination criterion to the feature amount.

In a fifth aspect, the peak candidate may include a point in time at which the maximum value that satisfies the determination criterion has been acquired, and the specifying unit may specify the at least one first peak based on no less than a predetermined number of peak candidates that are present at the same point in time. According to the fifth aspect, it is possible to detect blood pressure surges by integrating peak candidates that are represented by points in time at which the maximum value that satisfies the determination criterion has been acquired.

In a sixth aspect, the specifying unit may narrow down the at least one first peak using another feature amount that is based on at least one of a waveform, time information, and frequency information regarding the time-series data. According to the sixth aspect, it is possible to prevent peak data from increasing, and it is possible to appropriately detect an instance that can be regarded as a surge.

In a seventh aspect, the other feature amount may be a rise time, a fall time, an area, or a correlation coefficient of a blood pressure surge.

An eight aspect is the blood pressure data processing apparatus according to one of the first to seventh aspects, further including a display unit configured to display the at least one first peak together with the time-series data.

In a ninth aspect, the blood pressure data processing apparatus may include a search unit configured to detect at least one second peak by searching for a local maximum value of the time-series data at at least one of a time point that precedes a search range that includes the at least one first peak and a time point that is subsequent to the search range.

According to the ninth aspect, the local maximum value of the time-series data is searched for. Therefore, compared to the case in which only the first peak is specified, it is possible to detect more peaks. Also, according to the fifth aspect, it is possible to detect blood pressure surges as a second peak that precedes the first peak and a second peak that is subsequent to the first peak.

In a tenth aspect, the blood pressure data processing apparatus may include: a display unit that displays the at least one first peak and the at least one second peak together with the time-series data; and a display control unit configured to control the display unit so as to display the at least one first peak and the at least one second peak so as to be distinguishable from each other. According to the tenth aspect, it is possible to meet the wish of a user to check the result of detection of peaks occurred in a relatively long period, i.e. a relatively long blood pressure surge, as well as the wish of the user to check the detailed result of detection of peaks, i.e. a blood pressure surge occurred before or after a relatively long blood pressure surge and detected through the above-described search.

According to the present invention, it is possible to provide a technique for detecting a blood pressure surge from time-series data regarding the blood pressure value.

### BRIEF DESCRIPTION OF DRAWINGS

FIG. 1 is a block diagram showing a blood pressure data processing apparatus according to a first embodiment.

FIG. 2 s a block diagram showing an example of the blood pressure measurement apparatus shown in FIG. 1.

FIG. 3 is a side view showing a blood pressure measurement unit shown in

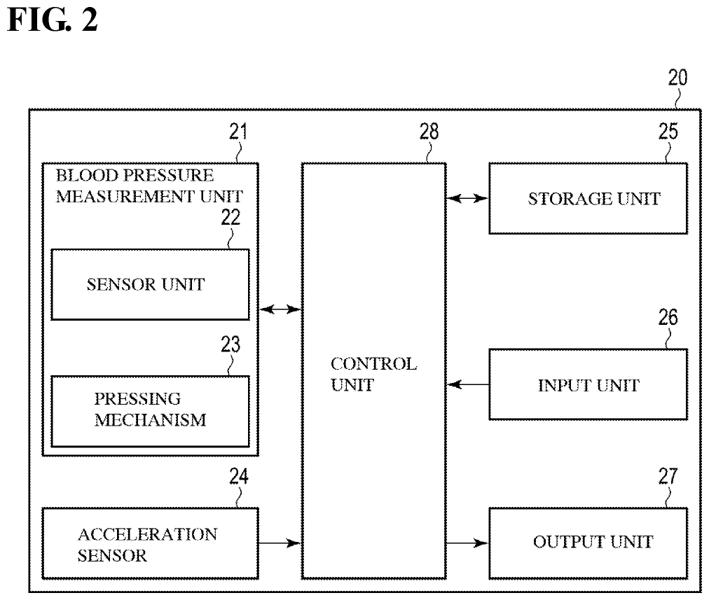

FIG. 2.

FIG. 4 is a cross-sectional view showing the blood pressure measurement unit shown in FIG. 2.

FIG. 5 is a plan view showing the blood pressure measurement unit shown in

FIG. 2.

FIG. 6 is a diagram showing a waveform of a pressure measured by each pressure sensor shown in FIG. 5.

FIG. 7 is a diagram showing an example of a slide window.

FIG. 8 is a flowchart showing an example of a processing procedure for outputting data regarding a first peak.

FIG. 9 is a diagram showing an example of spike noise removal.

FIG. 10 is a diagram showing an example of large-fluctuation noise removal.

FIG. 11 is a flowchart showing the details of the repetitive processing shown in FIG. 8.

FIG. 12 is a diagram showing the result of detection of blood pressure surges performed by the blood pressure data processing apparatus according to the first embodiment.

FIG. 13 is a block diagram showing a blood pressure data processing apparatus according to a second embodiment.

FIG. 14 is a flowchart showing an example of a processing procedure for outputting data regarding a second peak.

FIG. 15A is a diagram showing a surge that occurs in a relatively short period of time.

FIG. 15B is a diagram showing a surge that occurs in a relatively long period of time.

FIG. 16 is a diagram showing an example of an undetected surge.

FIG. 17A is a diagram showing a search for a maximum local maximum value at a point in time that precedes a surge point.

FIG. 17B is a diagram showing a search for a maximum local maximum value at a point in time that is subsequent to a surge point.

FIG. 18 is a block diagram showing a blood pressure data processing apparatus according to a third embodiment.

FIG. 19 is a diagram showing an example of display performed by a visualization unit.

FIG. 20 is a diagram showing an example of a visualization file output from the visualization unit.

FIG. 21 is a block diagram showing an example of a hardware configuration of a blood pressure data processing apparatus.

### DESCRIPTION OF EMBODIMENTS

The following describes embodiments of the present invention with reference to the drawings. In the following embodiments, parts that are assigned with the same reference numerals perform the same operations, and redundant descriptions are omitted.

### First Embodiment

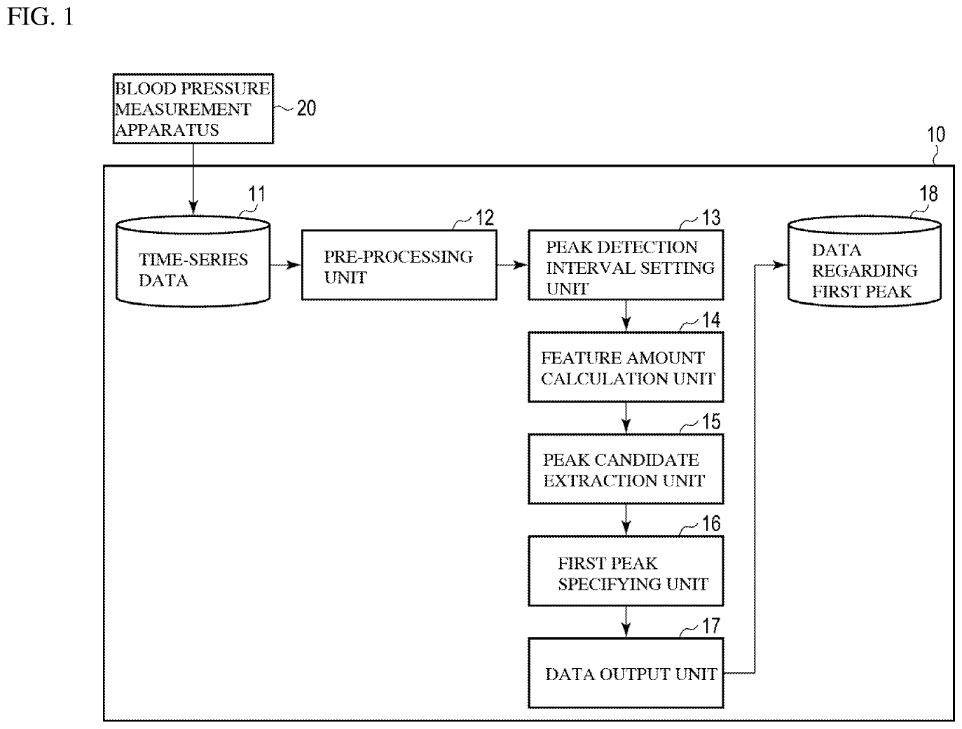

FIG. 1 schematically shows a blood pressure data processing apparatus 10 according to a first embodiment of the present invention. As shown in FIG. 1, the blood pressure data processing apparatus 10 processes time-series data 11 regarding a blood pressure value acquired by a blood pressure measurement apparatus 20 that measure the blood pressure of a measurement subject. The blood pressure data processing apparatus 10 may be implemented on a computer such as a personal computer or a server.

First, the blood pressure measurement apparatus 20 will be described with reference to FIGS. 2 to 6. In the first embodiment, the blood pressure measurement apparatus 20 is a wearable apparatus to be attached to a wrist of the measurement subject, and measures the pressure pulse wave of the radial artery through tonometry. Here, “tonometry” refers to a method in which a flat portion is formed in the artery by pressing the artery with an appropriate pressure from above the skin, and a pressure pulse wave is non-invasively measured using a pressure sensor in a state in which the interior and exterior of the artery are balanced. According to tonometry, it is possible to acquire a blood pressure value for each heartbeat.

FIG. 2 schematically shows the blood pressure measurement apparatus 20 according to the first embodiment. As shown in FIG. 2, the blood pressure measurement apparatus 20 includes a blood pressure measurement unit 21, an acceleration sensor 24, a storage unit 25, an input unit 26, an output unit 27, and a control unit 28. The control unit 28 controls the units of the blood pressure measurement apparatus 20. The function of the control unit 28 can be realized by a processor such as a CPU (Central Processing Unit) executing a control program stored in a computer-readable storage medium such as a ROM (Read-Only Memory).

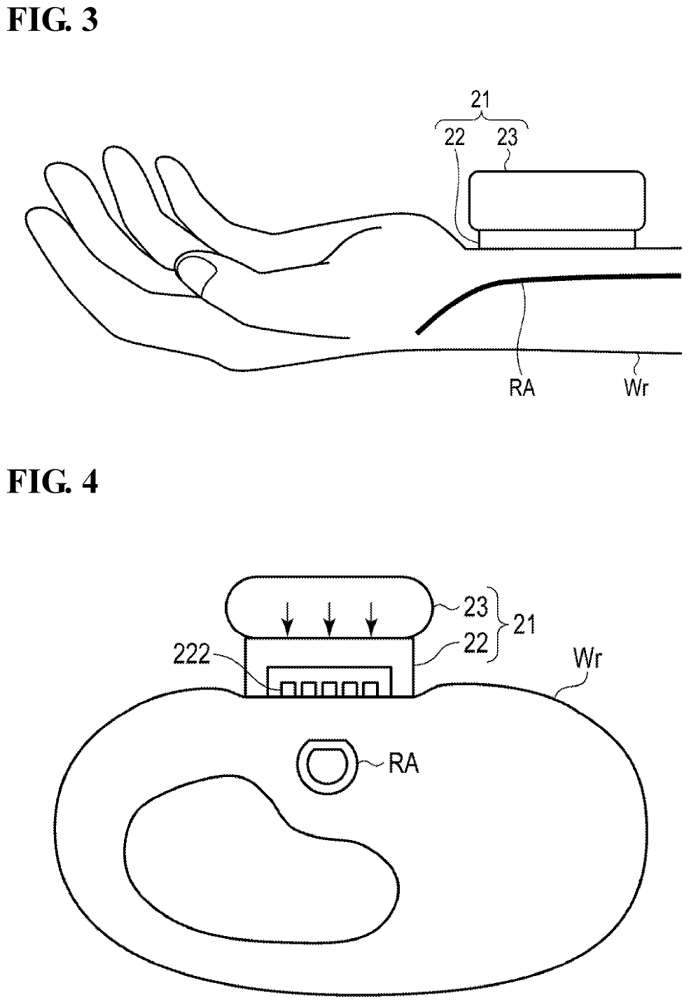

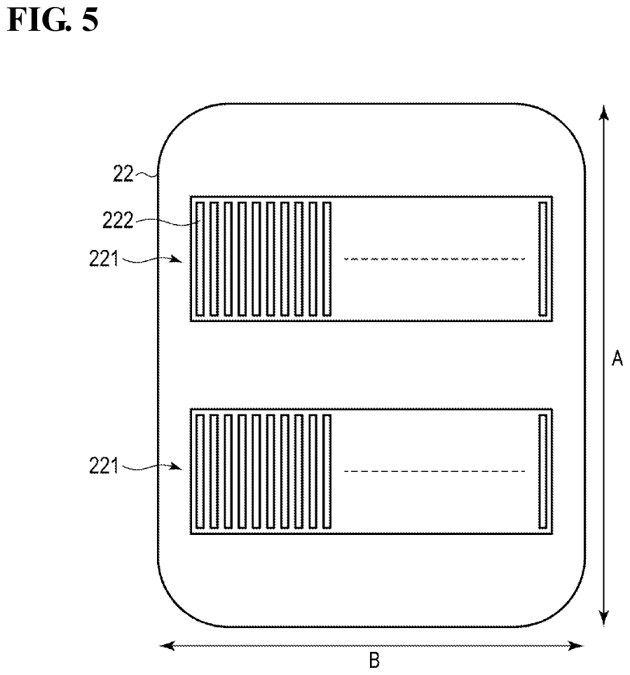

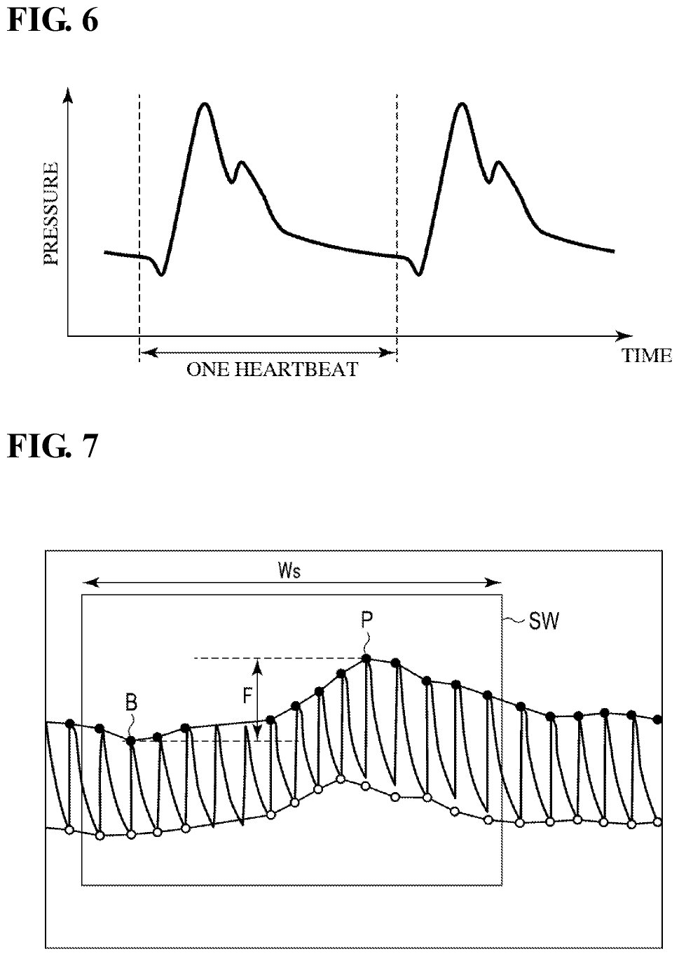

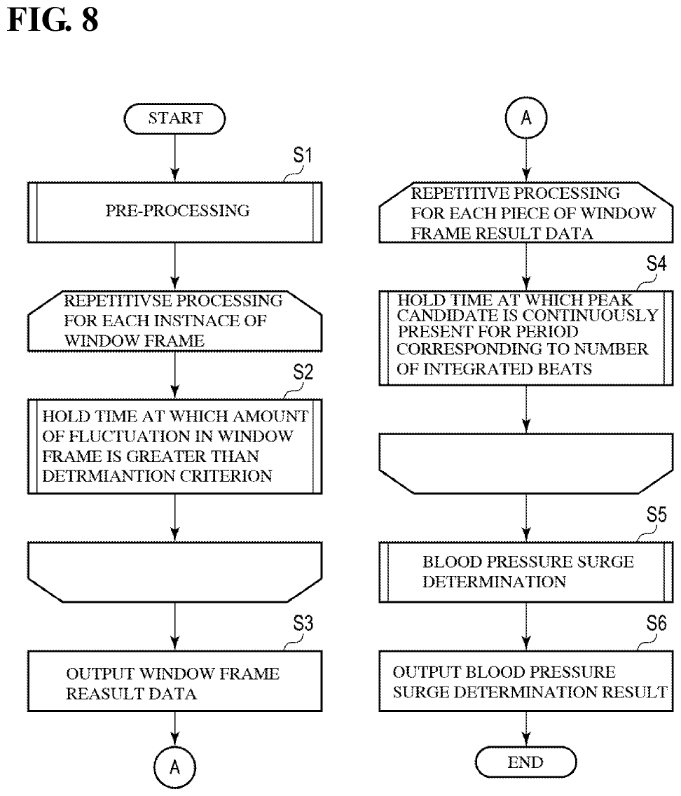

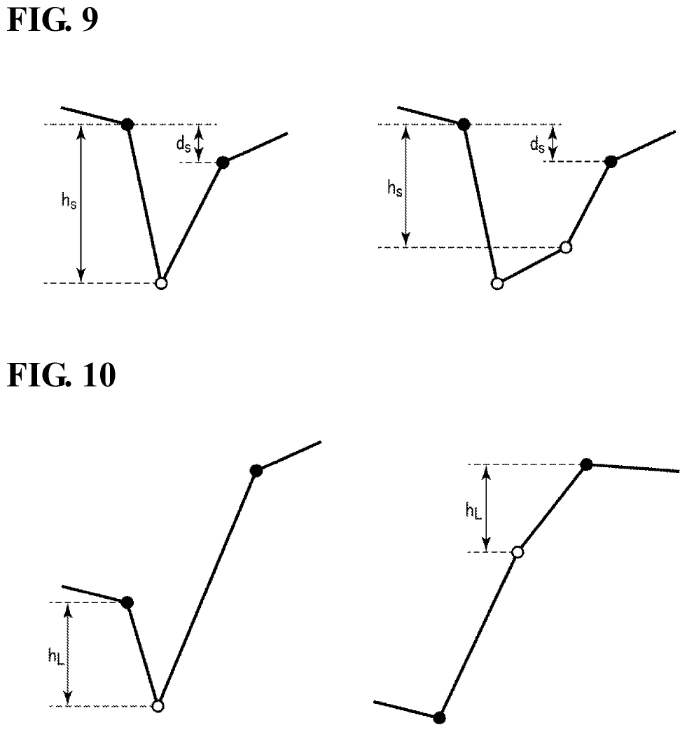

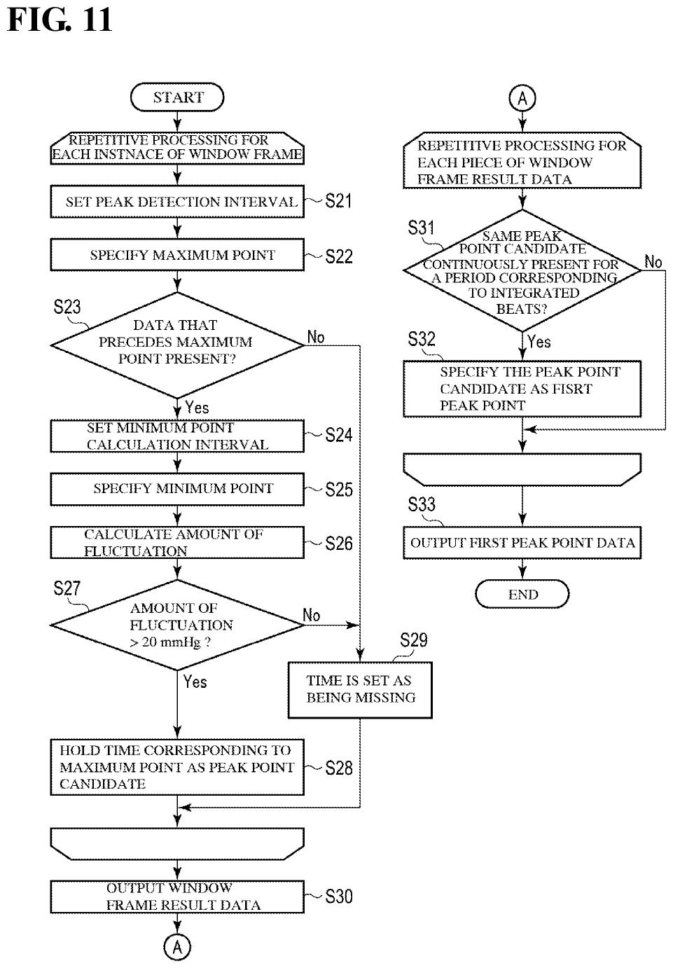

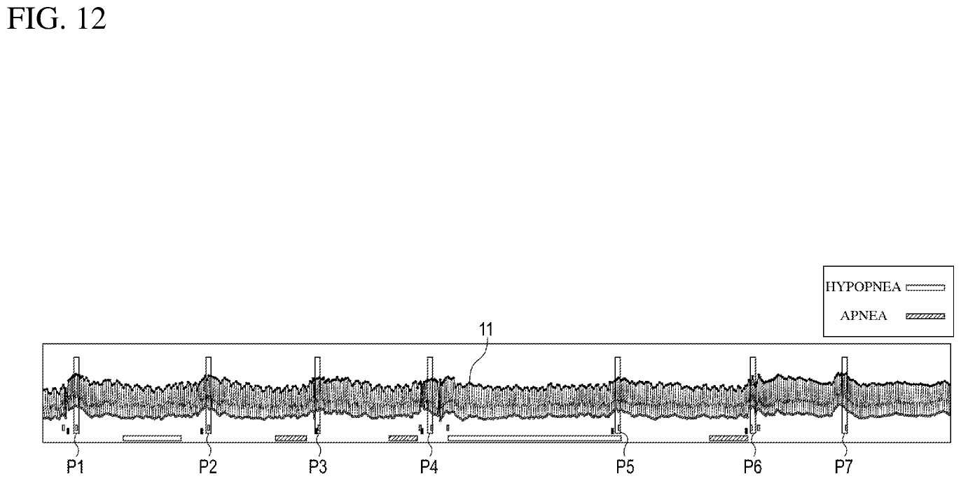

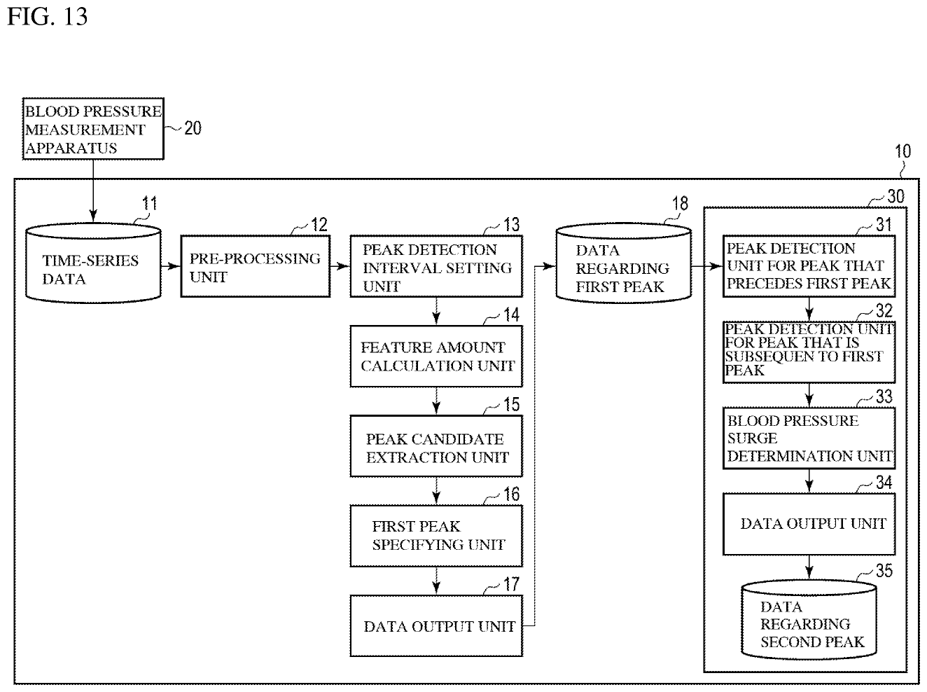

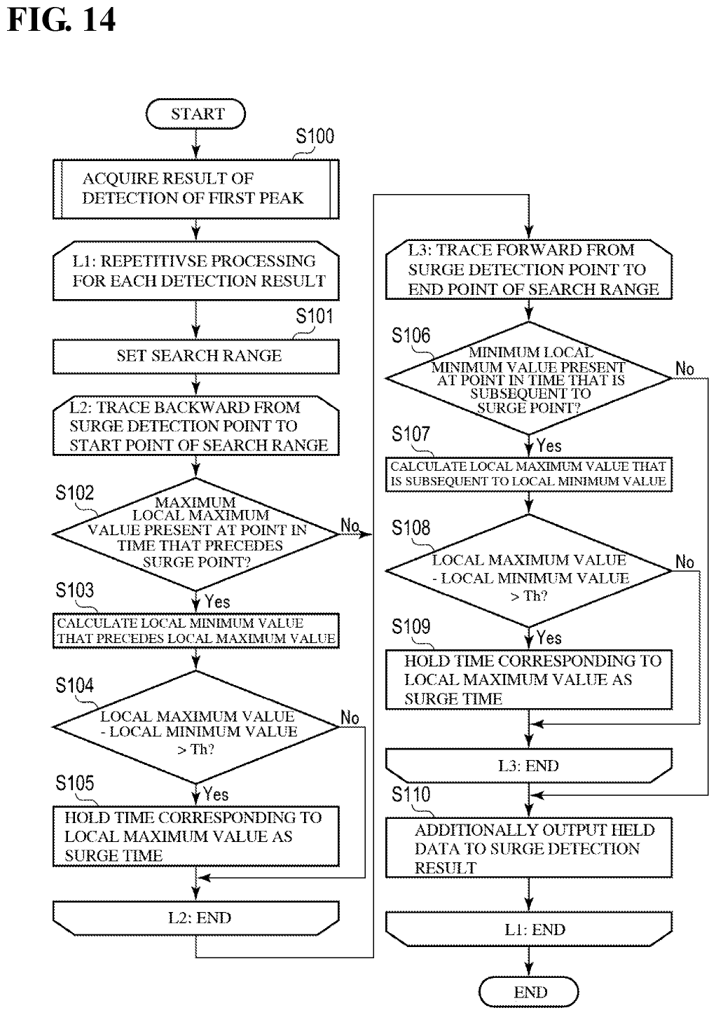

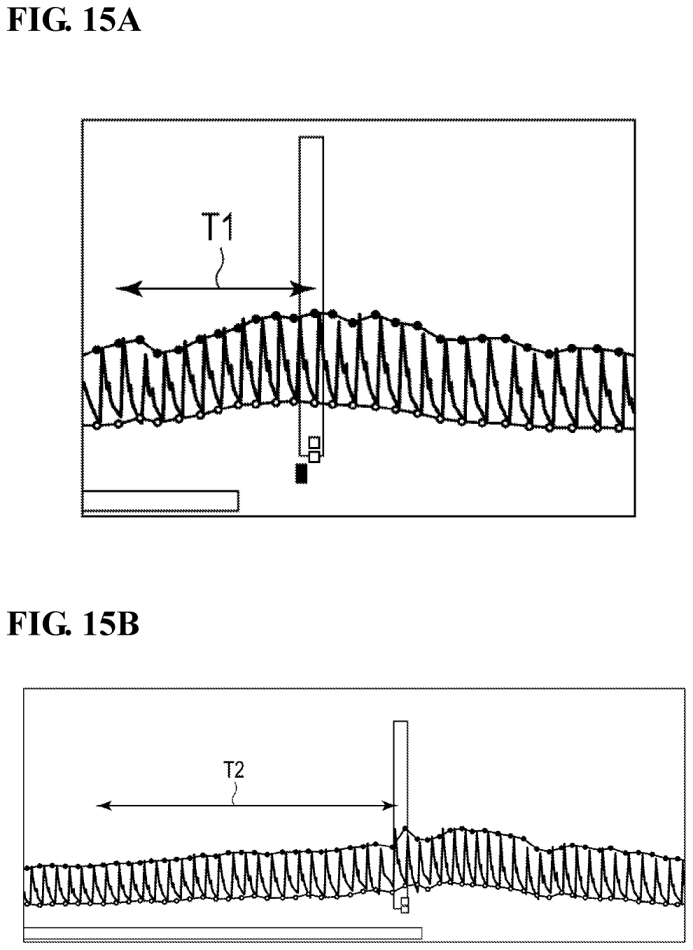

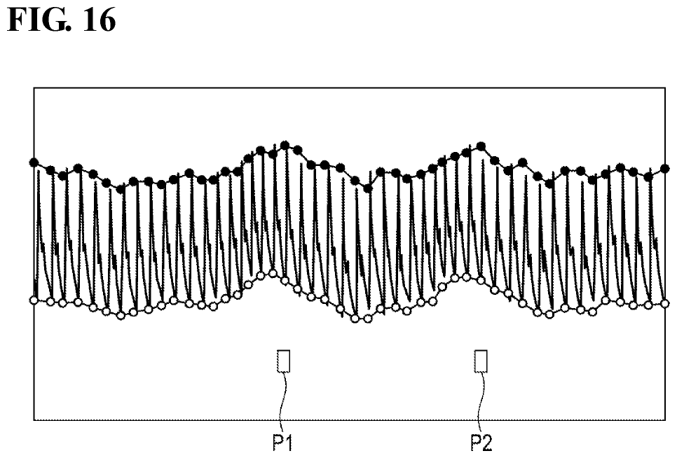

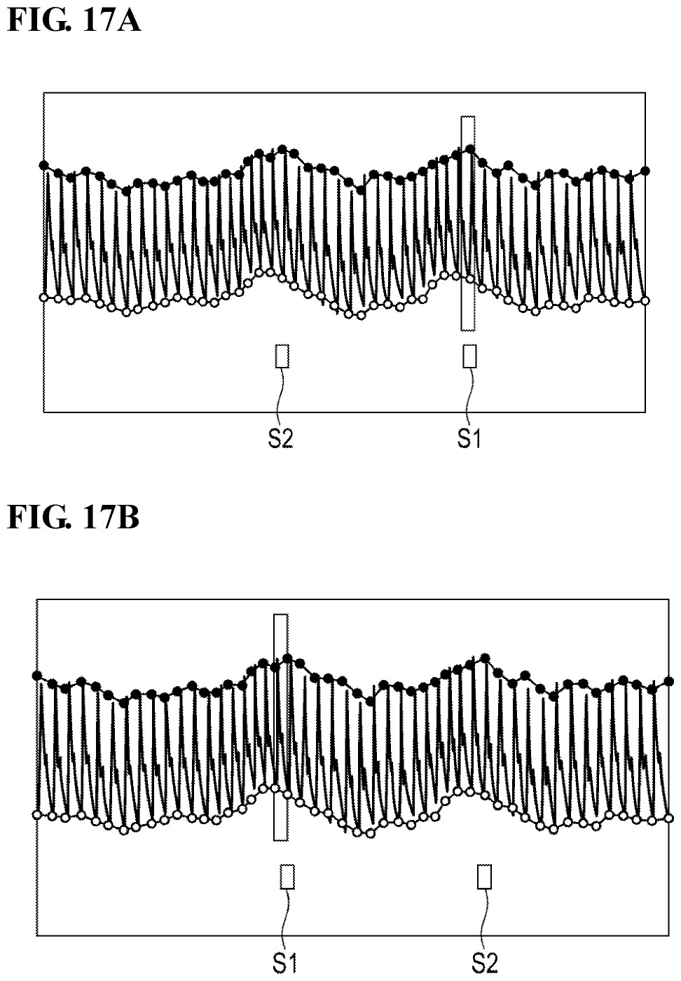

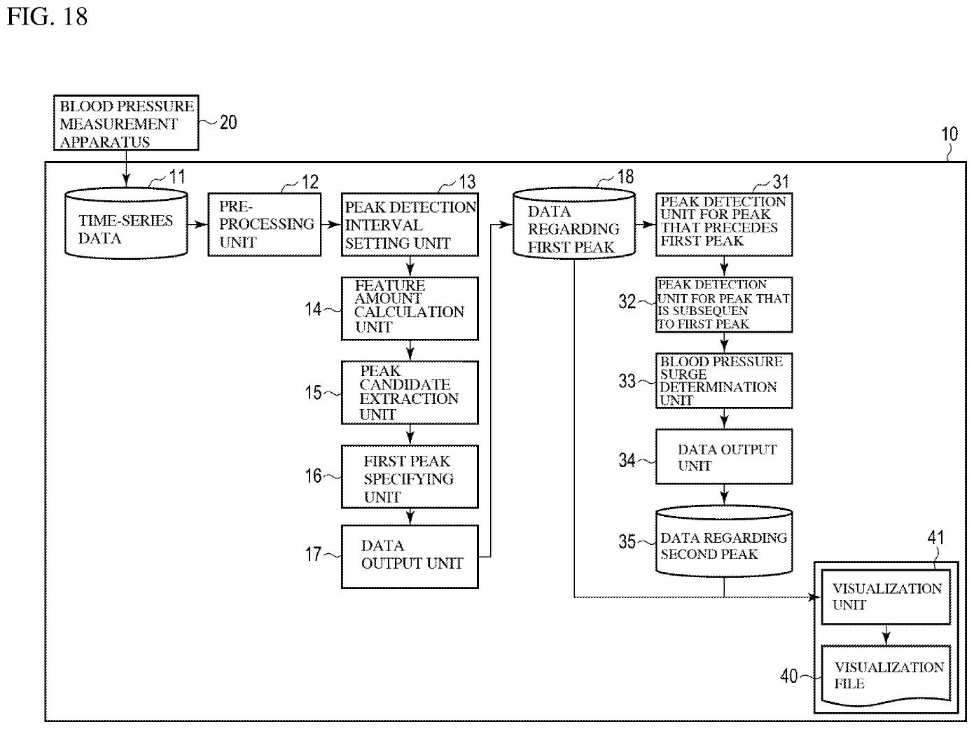

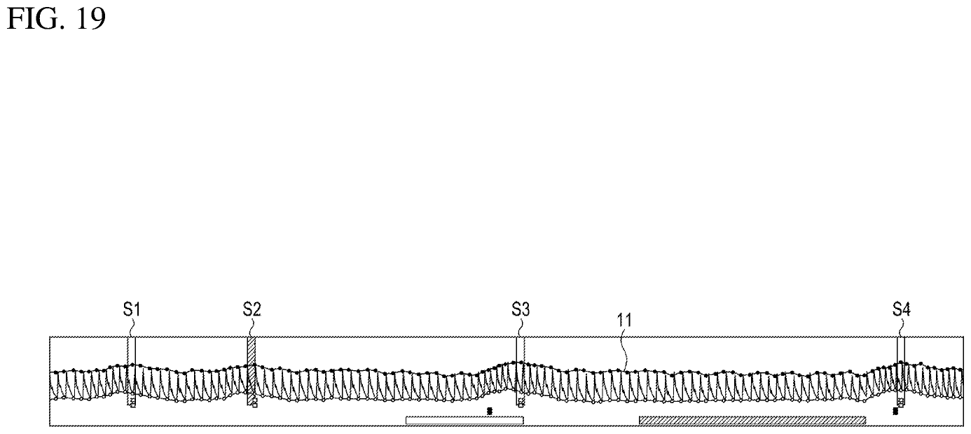

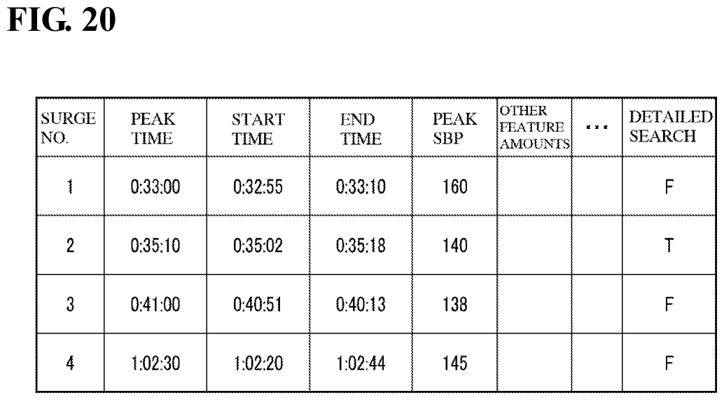

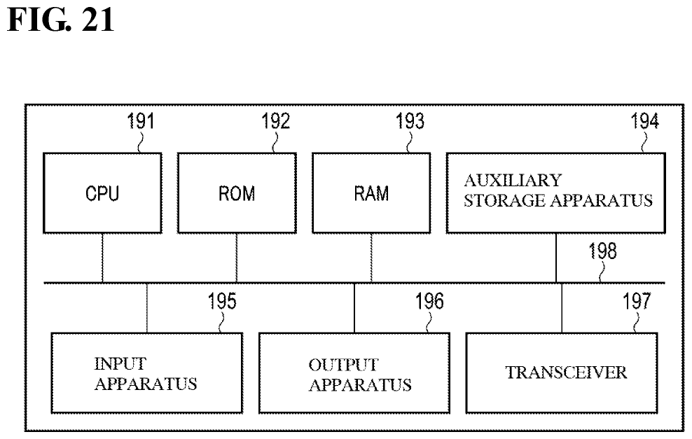

The blood pressure measurement unit 21 measures the pressure pulse wave of a measurement subject, and generates blood pressure data that include the result of measurement of the pressure pulse wave. FIG. 3 is a side view showing a state in which the blood pressure measurement unit 21 is attached to a wrist Wr of the measurement subject using a belt (not shown), and FIG. 4 is a cross-sectional view schematically showing a structure of the blood pressure measurement unit 21. As shown in FIGS. 3 and 4, the blood pressure measurement unit 21 includes a sensor unit 22 and a pressing mechanism 23. The sensor unit 22 is arranged so as to come into contact with a part (in this example, the wrist Wr) in which the radial artery RA is present. The pressing mechanism 23 presses the sensor unit 22 to the wrist Wr.

FIG. 5 shows a surface of the sensor unit 22 that comes into contact with the wrist Wr. As shown in FIG. 5, the sensor unit 22 includes one or more (in this example, two) pressure sensor arrays 221, and each pressure sensor array 221 has multiple (e.g., 46) pressure sensors 222 that are aligned in a direction B. The direction B is a direction that intersects a direction A in which the radial artery extends in a state in which the blood pressure measurement apparatus 20 is attached to the measurement subject. The pressure sensors 222 are assigned channel numbers. The arrangement of the pressure sensors 222 is not limited to the example shown in FIG. 5.

The pressure sensors 222 generate pressure data by measuring the pressure. Piezoelectric elements that convert pressure into electric signals can be used as the pressure sensors. The sampling frequency is 125 Hz, for example. A pressure waveform such as that shown in FIG. 6 is acquired as pressure data. The result of measurement of the pressure pulse wave is generated based on the pressure data output from one pressure sensor (active channel) 222 selected adaptively from among the pressure sensors 222. The maximum value of the waveform of the pressure pulse wave of one heartbeat corresponds the systolic blood pressure (SBP), and the minimum value of the waveform of the pressure pulse wave of one heartbeat corresponds to the diastolic blood pressure (DBP). Blood pressure data may include the pressure data output from the pressure sensors 222, as well as the result of measurement of the pressure pulse wave. Note that the result of measurement of the pressure pulse wave may be generated by the blood pressure data processing apparatus 10 based on the pressure data instead of being generated by the blood pressure measurement apparatus 20.

For example, the pressing mechanism 23 includes an air bag and a pump for adjusting the internal pressure of the air bag. When the pump is driven by the control unit 28 so as to increase the internal pressure of the air bag, the pressure sensor 222 is pressed to the wrist Wr due to the inflation of the air bag. Note that the pressing mechanism 23 is not limited to a structure using an air bag, and may also be realized by any structure in which the force pressing the pressure sensors 222 to the wrist Wr can be adjusted.

The acceleration sensor 24 detects the acceleration acting on the blood pressure measurement apparatus 20 and generates acceleration data. For example, a triple-axial acceleration sensor can be used as the acceleration sensor 24. The detection of the acceleration is carried out in parallel with the blood pressure measurement.

The storage unit 25 includes a computer-readable storage medium. For example, the storage unit 25 includes a ROM, a RAM (Random Access Memory) and an auxiliary storage apparatus. The ROM stores the above-described control program. The RAM is used as a work memory by the CPU. The auxiliary storage apparatus stores various types of data including the blood pressure data generated by the blood pressure measurement unit 21, and the acceleration data generated by the acceleration sensor 24. The auxiliary storage apparatus includes a flash memory, for example. The auxiliary storage apparatus includes one or both of a storage medium built into the blood pressure measurement apparatus 20 and a removable medium such as a memory card.

The input unit 26 receives an instruction from the measurement subject. For example, the input unit 26 includes an operation button, a touch panel, and the like. The output portion 27 outputs information such as a pressure measurement result. For example, the output unit 27 includes a display apparatus such as a liquid crystal display apparatus.

The blood pressure measurement apparatus 20 with the above-described configuration outputs measurement data that includes the blood pressure data and the acceleration data.

Next, blood pressure surge detection performed by the blood pressure data processing apparatus 10 according to the present embodiment will be described.

In the first embodiment, the blood pressure data processing apparatus 10 processes time-series data 11 regarding the blood pressure value that is based on the measurement data acquired from the blood pressure measurement apparatus 20, to output data 18 regarding a first peak of blood pressure surges. Although the value of the systolic blood pressure (SBP) is used as the time-series data 11 in the present embodiment, there is no limitation to this. Other values that can be used to observe blood pressure surges may be used as the time-series data 11 regarding the blood pressure value. For example, the diastolic blood pressure (DBP) or the pulse pressure (PP) may be used.

The blood pressure data processing apparatus 10 according to the present embodiment applies a slide window to the time-series data 11 regarding the blood pressure value per beat to identify the peak of a blood pressure surge. Note that the time-series data 11 need not be blood pressure value data acquired strictly at each beat. Also, although a “slide window” is also referred to as a “window frame” in the following description, these terms are used in the same meaning.

The peak of a blood pressure surge output from the blood pressure data processing apparatus 10 according to the first embodiment is referred to as a “first peak”, and the peak of a blood pressure surge output from the blood pressure data processing apparatus 10 according to the second embodiment described below is referred to as a “second peak”. Differences between the first peak and the second peak will be described in the second embodiment.

FIG. 7 shows an example of the slide window applied to the time-series data 11 regarding the blood pressure value. The slide window SW shown in the figure moves (slides) along the time axis at each beat. The width of movement along the time axis corresponds to one beat, for example. The slide window SW has a constant window width Ws along the time axis. The window width Ws is equal to the length of fifteen beats, for example. The window width Ws corresponds to the length of a peak detection interval when a candidate of the peak of the blood pressure value is extracted from each instance of the slide window SW that slides. FIG. 7 shows a waveform of the time-series data 11 regarding the blood pressure value included in the slide window SW at a given point in time. Whether or not a portion of the time-series data 11 in the slide window SW is a blood pressure surge is determined based on the feature amount of the blood pressure value.

The feature amount is, for example, a difference F between a point P (also referred to as a “maximum point”) at which the SBP in the slide window SW takes its maximum value, and a point B (also referred to as a “minimum point”) that precedes the point P in the slide window SW and at which the SBP takes its minimum value. Such a difference F is equal to the amount of fluctuation of the SBP in the slide window SW. Note that the feature amount is not limited to the amount of fluctuation of the SBP. Upon the feature amount being calculated for the slide window SW, determination is performed as to whether or not the feature amount satisfies a determination criterion.

A value that can be compared with the above-described difference F of the SBP is used as the determination criterion. For example, the determination criterion is 20 mmHg. The determination criterion value is not limited to this value. For example, the determination criterion may be 15 mmHg. If the determination criterion is satisfied, at least the time at the point P (i.e. the peak time of a surge) is held as the result of determination. The result of determination may include the start time of a surge, the end time of a surge, the SBP at the peak, and other feature amounts in addition to the peak time.

The results of determination regarding a plurality of instances of the slide window SW are stored in the memory as the respective peak candidates of peak detection intervals. The results of determination regarding a plurality of instances of the slide window SW that slides along the time axis, i.e. the respective peak candidates of the peak detection intervals, are integrated, and at least one first peak is specified. Specifically, if no less than a predetermined number of peak candidates have been acquired with respect to the same point in time, the point in time is determined as the time corresponding to the first peak. It is thought that the same peak is output from the instances of the slide window SW around the peak.

Here, the predetermined number is five, for example. In the present embodiment, in which time-series data is used in units of beats and the slide window SW slides at each beat, this predetermined number is referred to as the number of “integrated beats”. Note that the number of integrated beats is not limited to five, and is appropriately determined in view of the accuracy in detecting the peak and the processing speed.

Note that the above-described processing using the slide window SW may be modified as follows.

For example, the maximum point of the SBP may be determined as a peak candidate. If this is the case, the maximum point of the SBP is determined as a peak candidate as it is without comparing the amount of fluctuation of the SBP with the determination criterion in each kind of processing carried out while the slide window SW slides. Ultimately, the respective maximum points of the SBP in the instances of the slide window SW are integrated using the number of integrated beats, and thus the first peak is specified.

The following describes the configuration of the blood pressure data processing apparatus 10 according to the first embodiment.

As shown in FIG. 1, the blood pressure data processing apparatus 10 includes a pre-processing unit 12, a peak detection interval setting unit 13, a feature amount calculation unit 14, a peak candidate extraction unit 15, a first peak specifying unit 16, and a data output unit 17. Note that, if the maximum point of the SBP is determined as a peak candidate as it is without being compared with the determination criterion as in the above-described modification, the peak candidate extraction unit 15 may be omitted from the constituent elements. That is, the feature amount calculation unit 14 may output a peak candidate.

The blood pressure data processing apparatus 10 holds time-series data 11 regarding the blood pressure value that is based on measurement data acquired by the blood pressure measurement apparatus 20. A removable medium may be used to provide the time-series data 11 regarding the blood pressure value from the blood pressure measurement apparatus 20 to the blood pressure data processing apparatus 10. Alternatively, communication (wired communication or wireless communication) may be performed to provide the time-series data 11 regarding the blood pressure value from the blood pressure measurement apparatus 20 to the blood pressure data processing apparatus 10.

The pre-processing unit 12 performs pre-processing such as smoothing using a moving average or the like, noise removal, and high-frequency component removal using a low-pass filter, on the time-series data 11 regarding the blood pressure value acquired from the blood pressure measurement apparatus 20.

The peak detection interval setting unit 13 sets peak detection intervals to the time-series data 11 that has undergone the pre-processing performed by the pre-processing unit 12.

The feature amount calculation unit 14 calculates a feature amount that is based on one of the systolic blood pressure (SBP), the diastolic blood pressure (DBP), and the pulse pressure (PP) in each of the peak detection intervals set by the peak detection interval setting unit 13. For example, the feature amount calculation unit 14 calculates the difference F between the point P at which the SBP in the slide window SW takes its maximum value, and the point B that precedes the point P in the slide window SW and at which the SBP takes its minimum value.

The peak candidate extraction unit 15 applies the determination criterion to the feature amount calculated by the feature amount calculation unit 14 to extract a peak candidate from each peak detection interval. Note that the peak candidate extraction unit 15 may be configured to not perform any processing when the feature amount (the amount of fluctuation) is not to be compared with the determination criterion as in the above-described modification.

Upon the peak candidate extraction unit 15 extracting the respective peak candidates from the peak detection intervals, the first peak specifying unit 16 specifies at least one first peak from among the peak candidates. For example, if five or more peak candidates have been acquired with respect to the same point in time, the first peak specifying unit 16 determines the point in time as the time corresponding to the first peak.

The data output unit 17 outputs the data 18 regarding the first peak specified by the first peak specifying unit 16. The data 18 regarding the first peak includes the time corresponding to the first peak and the blood pressure value of the first peak at the time (the value of the SBP in the present embodiment).

Next, operations of the blood pressure data processing apparatus 10 according to the present embodiment will be described. FIG. 8 is a flowchart showing an example of a processing procedure for outputting data regarding the first peak.

In step S1, the pre-processing unit 12 performs pre-processing such as smoothing using a moving average or the like, noise removal, and high-frequency component removal using a low-pass filter on the time-series data 11 regarding the blood pressure value acquired from the blood pressure measurement apparatus 20.

FIG. 9 shows an example of spike noise removal, which is a type of noise removal. The time-series data 11 regarding the blood pressure value may include spike noise. A blood pressure value corresponding to a spike that has a large height h_{S} and a small difference d_{S} between the values at the ends thereof is removed through spike noise removal. For example, a blood pressure value that satisfies h_{S}≥13 (mmHg) and d_{S}≤7 (mmHg) is removed from the time-series data 11. In the example on the left in FIG. 9, the white circle represents one-point spike noise, which indicates a blood pressure value that is to be removed. In the example on the right in FIG. 9, the white circles represent two-point spike noise, which indicate blood pressure values that are to be removed. Note that spike noise with a waveform that is the inversion of the waveform of each example of spike noise shown in FIG. 9 may be removed. A data point from which a blood pressure value has been removed may be given an interpolation value that is calculated based on the blood pressure values of data points that come before and after the data point.

FIG. 10 shows an example of large-fluctuation noise removal. The time-series data 11 regarding the blood pressure value may include noise of a large fluctuation of the blood pressure value for a certain reason other than a blood pressure surge. In large-fluctuation noise removal, if the difference h_{L} between blood pressure values at points before and after a beat is no less than a predetermined value, the blood pressure value corresponding to the difference h_{L} is removed from the time-series data 11. For example, a blood pressure value that satisfies h_{L}≥20 (mmHg) regarding the amount of fluctuation is removed from the time-series data 11 as large-fluctuation noise. In the example on the left in FIG. 10, the white circle represents noise that is to be removed in a case where the blood pressure value has a trend to decrease, and in the example on the right in FIG. 10, the white circle represents noise that is to be removed in a case where the blood pressure value has a trend to increase. A data point from which a blood pressure value has been removed may be given an interpolation value that is calculated based on the blood pressure values of data points that come before and after the data point.

Next, repetitive processing is performed for each window frame. The window frame moves along the time axis at each beat. In step S2, the time at which the amount of fluctuation in the window frame exceeds the determination criterion is held. Specifically, the feature amount calculation unit 14 calculates a feature amount that is based on one of the systolic blood pressure, the diastolic blood pressure, and the pulse pressure in the peak detection intervals set by the peak detection interval setting unit 13. If the feature amount is greater than the determination criterion (20 mmHg in this example), the peak candidate extraction unit 15 holds the time corresponding to the maximum point as a peak candidate. Step S2 is repeatedly executed while the window frame is moved along the time axis. While the window frame moves (slides), the peak detection interval setting unit 13 sets peak detection intervals by shifting a beat position to the position of the next beat. The processing in Step S2 is repeatedly performed up to the position of the last beat in the time-series data 11, and window frame result data is ultimately output (step S3).

Next, in order to specify the first peak, repetitive processing is performed on the window frame result data output in step S3. In step S4, if five or more peak candidates have been acquired with respect to the same point in time in the window frame result data, the first peak specifying unit 16 determines the point in time as the time corresponding to the first peak. Step S4 is executed for all of the pieces of window frame result data. Ultimately, all of the points in time (i.e. first peaks) at which a peak candidate is continuously present for a period corresponding to the number of integrated beats are specified.

Next, surge determination is performed in step S5. In this step, the results of first peak detection are narrowed down. The first peak specifying unit 16 narrows down the results of first peak detection according to another feature amount that is based on at least one of the waveform, time information, and frequency information of the time-series data 11. The other feature amount includes the rise time, fall time, area, and correlation coefficient of a blood pressure surge.

For example, the first peaks may be narrowed down such that, if two first peaks that are close to each other have been detected and the SBP values thereof are approximately the same, the first peak with the higher SBP value is adopted and the other first peak is not adopted. The condition regarding the minimum point (the surge start point) that is used by the feature amount calculation unit 14 to calculate the feature amount (the amount of fluctuation) may be given a greater weight. Specifically, instead of the minimum value of the SBP, a point at which the blood pressure value is stable may be determined as the surge start point. If this is the case, it is possible to more accurately extract an instance that can be regarded as a surge. Furthermore, a correlation coefficient that indicates a trend to increase from the surge start point to the maximum point may be calculated, and the results of first peak detection may be narrowed down based on the correlation coefficient thus calculated. Specifically, a relationship between the time from the serge start point to the maximum point and the SBP may be calculated as a correlation coefficient, and a first peak may be determined as a surge if the correlation coefficient is greater than a predetermined threshold value, and a first peak may be determined as a non-surge if the correlation coefficient is no greater than the predetermined threshold value. Such surge determination may be performed using another available feature amount such as the feature amounts of the SBP and the DBP, and the feature amount of the pressure pulse wave (e.g. data recorded at 125 Hz).

Thereafter, in step S6, the data 18 regarding the first peak is output from the data output unit 17 as the result of blood pressure surge detection.

Note that, through the processing shown in FIG. 8, repetitive processing is performed in step S2 to compare the amount of fluctuation in the window frame with the determination criterion, and repetitive processing is performed in step S4 to integrate peak candidates corresponding to the same point in time, and thus surge determination is performed (batch processing). However, surge determination may be performed through real-time processing, through which the afore-mentioned two types of repetitive processing are approximately simultaneously performed.

FIG. 11 is a flowchart showing the details of the repetitive processing shown in FIG. 8. In steps S21 to S28, repetitive processing is performed for each instance of the window frame. These processing steps show further details of step S2 in FIG. 8. First, the window frame that is to be subjected to repetitive processing this time, i.e. the peak detection interval, is set (step S21). In the present embodiment, the length of the peak detection interval is fifteen beats, which is equal to the width of the window frame. Next, the maximum point at which the SBP takes its maximum value in the window frame that is to be processed is specified from the time-series data 11 regarding the blood pressure value (step S22). Subsequently, determination is performed as to whether or not data is present at a point that precedes the maximum point in the peak detection interval (step S23). If it is determined that data is present at a point that precedes the maximum point, processing proceeds to step S24. If it is determined that data is not present, processing proceeds to step S29.

If data is present at a point that precedes the maximum point, a minimum point calculation interval is set in the peak detection interval that is to be processed this time (step S24), and the minimum point of the SBP in the interval is specified (step S25). The amount of fluctuation of the SBP in the window frame that is to be processed is calculated based on the maximum point of the SBP specified in step S22 and the minimum point of the SBP specified in step S25 (step S26). The amount of fluctuation is expressed as SBP(max_time)−SBP(min_time), for example. Such an amount of fluctuation of the SBP is the amount of fluctuation in the window frame that is to be processed in the time-series data 11 regarding the blood pressure value.

Next, determination is performed as to whether or not the amount of fluctuation calculated in step S26 is greater than 20 (mmHg), which is the determination criterion (step S27). If the amount of fluctuation is greater than 20 (mmHg), processing proceeds to step S28. If the amount of fluctuation is no greater than 20 (mmHg), processing proceeds to step S29. In step S28, the time corresponding to the maximum point of the SBP is held in the memory as a candidate of the first peak point, and processing returns to step S21. In step S21, the window frame that is to be processed is updated, i.e. the peak detection interval is shifted to the position of the next beat, and the processing in step S22 and the subsequent steps is performed.

Note that, if the above-described modification in which comparison with the determination criterion is not performed and the maximum value of the SBP is determined as it is as a peak candidate is employed, steps S23 to S27 are skipped. Alternatively, the amount of fluctuation may be calculated through steps S23 to S26, and the determination criterion may be set to 0 (mmHg) for the sake of convenience in step S27 so that processing forcibly proceeds to step S28.

In step S29, the time is set as being missing. I other words, it is determined that no candidate of the first peak point can be acquired, and the window frame that is to be processed is updated to the next window frame.

Upon processing being complete up to the last window frame, window frame result data is output (step S30). Window frame result data includes the value of the SBP that is a candidate of the first peak point, and the time corresponding to the candidate of the first peak point.

Subsequently, in steps S31 to S33, repetitive processing is performed for each piece of window frame result data. These processing steps show further details of step S4 in FIG. 8. In this example, determination is performed as to whether or not the first peak point candidate is continuously present at the same point of time for a period corresponding to the integrated beats (step S31). The number of integrated beats is five in the present embodiment. If it is determined that the first peak point candidate is continuously present at the same point of time for the period corresponding to the integrated beats, the first peak point candidate is determined as the first peak point (step S32). In step S31, if it is determined that the first peak point candidate at the same point in time is not continuously present for the period corresponding to the integrated beats, step S32 is skipped and the same processing is repeated for the next window frame result data. Upon processing being complete up to the last window frame result data, the first peak point data is output (step S33). The first peak point data is the data 18 regarding the first peak shown in FIG. 1, and include the value of the SBP at the first peak point and the time corresponding to the first peak point.

FIG. 12 is a diagram showing the result of detection of blood pressure surges performed by the blood pressure data processing apparatus 10 according to the first embodiment. The figure shows a plurality of first peak points P1 to P7 detected by the blood pressure data processing apparatus 10 according to the first embodiment as blood pressure surges, as well as the waveform of the time-series data 11 regarding the blood pressure value.

Blood pressure surges are characterized in that they do not necessarily periodically occur, and the amount of an increase and the rise time of the blood pressure value is various. According to the present embodiment, it is possible to detect such blood pressure surges.

According to the above-described first embodiment, it is possible to specify the first peak of the blood pressure value by integrating a plurality of peak candidates that satisfy the determination criterion in the time-series data 11 regarding the blood pressure value. Therefore, it is possible to detect blood pressure surges as the first peaks. Also, according to the first embodiment, it is possible to accurately detect blood pressure surges based on the time-series data 11 regarding the blood pressure value at each beat, and it is possible to robustly detect blood pressure surges that do not periodically occur and blood pressure surges that have various patterns. The feature amount that is used to detect surges is the difference between the maximum value of the SBP in a peak detection interval and the minimum value of the SBP at a point that precedes the maximum value in the peak detection interval. Therefore, it is possible to detect blood pressure surges where the blood pressure value sharply increases, based on the amount of fluctuation of the maximum value of the SBP in the peak detection interval.

### Second Embodiment

FIG. 13 is a block diagram showing a blood pressure data processing apparatus according to a second embodiment. The blood pressure data processing apparatus 10 according to the second embodiment is formed by adding a search unit 30 to the constituent elements of the blood pressure data processing apparatus 10 according to the first embodiment. The search unit 30 includes a peak detection unit 31 for a peak that precedes the first peak, a peak detection unit 32 for a peak that is subsequent to the first peak, a blood pressure surge determination unit 33, and a data output unit 34.

The search unit 30 searches the time-series data 11, which represents the first peak, for the second peak corresponding to a blood pressure surge. Data 35 regarding the second peak is output as a result of search processing.

According to the first embodiment, the data 18 regarding the first peak is output based on the time-series data 11 regarding the blood pressure value.

Specifically, the slide window is applied to the time-series data 11, the amount of fluctuation of the SBP is calculated for each instance of the window frame, the amount of fluctuation is compared with the determination criterion for blood pressure surges, a plurality of determination results including candidates of the first peak are integrated for each instance of window frame to specify the first peak, and the data 18 regarding at least one first peak is output.

In contrast, according to the second embodiment, the search unit 30 is configured to search for the local maximum value of the blood pressure value data at at least one of a point in time that precedes the search are including the first peak or a point in time that is subsequent to the same in the time-series data 11 of the blood pressure value, thereby detecting at least one second peak. According to the above-described second embodiment, the local maximum value is searched for. Therefore, compared to the case in which only the first peak is specified, it is possible to detect more peaks, and detect blood pressure surges as a second peak that precedes the first peak and a second peak that is subsequent to the first peak.

Next, operations of the blood pressure data processing apparatus 10 according to the second embodiment will be described. FIG. 14 is a flowchart showing an example of a processing procedure for outputting data regarding a second peak.

In step S100, the search unit 30 acquires the data 18, which is the result of detection of the first peak. Preferably, the width of the window frame that is used to detect the first peak is set to be large enough to detect various types of surges. There are various patterns of blood pressure surges such as a surge that occurs in a relatively short period T1 (e.g. 10 seconds) as shown in FIG. 15A and a surge that occurs in a relatively long period T2 (e.g. 25 seconds) as shown in FIG. 15B. Therefore, it is difficult to define a template for detection. If the width of the window frame is increased to detect a long blood pressure surge, and if surges P1 and P2 occur at a relatively short interval as shown in FIG. 16, only one of the surges can be detected. According to the second embodiment, it is possible to detect the second peak through a search for a local maximum value that precedes the first peak and a local maximum value that is subsequent to the first peak, even if the width of the window frame that is used to detect the first peak is sufficiently increased.

Next, the search unit 30 performs repetitive processing L1 for each result of detection of the first peak. In repetitive processing L1, first, in step S101, the search unit 30 sets a range in which the second peak is to be searched for, regarding the first peak that is to be processed in the current repetitive processing L1, i.e. a surge detection point. Next, the peak detection unit 31 for a peak that precedes the first peak performs repetitive processing L2. Through this processing, the peak detection unit 31 detects the local maximum value by tracing backward from the surge detection point to be processed to the start point of the search range set in step S101. Specifically, first, in step S102, determination is performed as to whether or not the maximum local maximum value is present at a point in time that precedes the surge point. FIG. 17A shows a search for the maximum local maximum value at a point in time that precedes the surge point. A local maximum value S2 that precedes a surge point S1 is searched for. If the maximum local maximum value is not present, the peak detection unit 31 exits the repetitive processing L2. If it is determined in step S102 that the maximum local maximum value is present, a local minimum value that precedes the local maximum value is calculated in step S103. Next, in step S104, the blood pressure surge determination unit 33 determines whether or not the difference between the local maximum value found in step S102 and the local minimum value calculated in step S103 is greater than a threshold value Th. If the difference is greater than the threshold value Th, the blood pressure surge determination unit 33 holds the time at which the local maximum value was taken as a surge time (a second peak) (step S105). If the difference is no greater than the threshold value Th, step S105 is skipped and the repetitive processing L2 is continued.

Upon the repetitive processing L2 being complete, the peak detection unit 32 for a peak that is subsequent to the first peak executes repetitive processing L3. Through this processing, the peak detection unit 32 detects the local maximum value by tracing forward along the time axis from the surge detection point to be processed to the end point of the search range set in step S101. FIG. 17B shows a search for the maximum local maximum value at a point in time that is subsequent to the surge point. A local maximum value S2 that is subsequent to the surge point S1 is searched for.

Specifically, first, in step S106, determination is performed as to whether or not the minimum local minimum value is present at a point in time that is subsequent to the surge point. If the minimum local minimum value is not present, the peak detection unit 32 exits the repetitive processing L3. If it is determined in step S106 that the minimum local minimum value is present, a local maximum value that is subsequent to the local minimum value is calculated in step S107. Next, in step S108, the blood pressure surge determination unit 33 determines whether or not the difference between the local maximum value found in step S107 and the local minimum value calculated in step S106 is greater than the threshold value Th. If the difference is greater than the threshold value Th, the blood pressure surge determination unit 33 holds the time at which the local maximum value was taken as a surge time (a second peak) (step S109). If the difference is no greater than the threshold value Th, step S109 is skipped and the repetitive processing L3 is continued.

In step S110, the data output unit 34 outputs the data 35 regarding the second peak as the surge time determined by the blood pressure surge determination unit 33. Thus, the data 35 regarding the second peak is output in addition to the data 18 regarding the first peak (the result of detection performed in step S100). The data 35 regarding the second peak may include the start time of a surge, the end time of a surge, the SBP at the peak, and other feature amounts in addition to the peak time.

As described above, according to the second embodiment, the local maximum value is searched for. Therefore, compared to the case in which only the first peak is specified, it is possible to detect more peaks, and detect blood pressure surges as a second peak that precedes the first peak and a second peak that is subsequent to the first peak. In other words, it is possible to detect surges that occur at short intervals relative to the width of the window frame.

### Third Embodiment

FIG. 18 is a block diagram showing a blood pressure data processing apparatus according to a third embodiment. The third embodiment is formed by adding a visualization unit 41 that outputs a visualization file 40, which is the result of detection of blood pressure surges, to the configuration of the blood pressure data processing apparatus 10 according to the second embodiment. The visualization unit 41 displays a blood pressure surge detected from the time-series data 11 as a first peak and a blood pressure surge detected by the search unit 30 according to the second embodiment as a second peak so as to be distinguishable from each other.

The visualization unit 41 may be added to the configuration of the blood pressure data processing apparatus 10 according to the first embodiment. In the first embodiment, the second peak is not detected, and the visualization unit 41 cannot display first peaks and second peaks so as to be distinguishable from each other. In a normal display mode, the visualization unit 41 displays first peaks detected as blood pressure surges on the time-series data 11.

In a normal display mode, the visualization unit 41 according to the third embodiment displays only first peaks detected as blood pressure surges, only second peaks, or both first peaks and second peaks on the time-series data 11 without distinguishing them from each other.

FIG. 19 shows an example of distinguishable display performed by the visualization unit 41. In the displayed waveform of the time-series data 11 regarding the blood pressure value, the blood pressure surges are displayed so as to be distinguishable from each other, indicating that the blood pressure surges S1, S3, and S4 have been detected as first peaks, and the blood pressure surge S2 has been detected as a second peak through search processing performed by the search unit 30. As described in the second embodiment, the width of the window frame that is employed in order to detect first peaks is set to be large enough to detect a long surge.

FIG. 20 shows an example of the visualization file 40 output from the visualization unit 41. The visualization file 40 includes column items indicating the surge No., the peak time, the start time, the end time, the peak SBP, and other feature amounts, and a column item (detailed search) indicating whether or not the record has been detected through a search, using a truth-value (T(rue)/F(alse)). For example, it is possible to extract only surges that have been detected through a search, by selecting “T” in the “detailed search” of the visualization file 40 and performing filtering processing.

According to the above-described third embodiment, it is possible to meet the wish of a user (i.e. an observer) to check the result of detection of first peaks occurred in a relatively long period, i.e. a relatively long blood pressure surge, as well as the wish of the user to check the detailed result of detection of peaks, i.e. a blood pressure surge occurred before or after a relatively long blood pressure surge and detected as a second peak through the above-described search. Although the blood pressure surges S1 to S4 in the example show in FIG. 20 are displayed at the same time, it is possible to employ a configuration with which display switching can be performed, e.g. the blood pressure S2 found through search processing is not displayed, or only the blood pressure surge S2 is displayed.

Next, an example of a hardware configuration of the blood pressure data processing apparatus 10 will be described with reference to FIG. 21.

The blood pressure data processing apparatus 10 includes a CPU 191, a ROM 192, a RAM 193, an auxiliary storage apparatus 194, an input apparatus 195, an output apparatus 196, and a transceiver 197, and these elements are connected to each other via a bus system 198. The above-described functions of the blood pressure data processing apparatus 10 can be realized by the CPU 191 reading out and executing a program stored in a computer-readable storage medium (the ROM 192 and/or the auxiliary storage apparatus 194). The RAM 193 is used as a work memory by the CPU 191. For example, the auxiliary storage apparatus 194 includes a hard disk drive (HDD) or a solid-state drive (SSD). The auxiliary storage apparatus 194 is used as a storage unit that stores the time-series data 11 shown in FIG. 1 and so on. The input apparatus includes, for example, a keyboard, a mouse, and a microphone. The output apparatus includes, for example, a display apparatus such as a liquid crystal display apparatus and a speaker. The transceiver 197 performs transmission and reception of signals to and from another computer. For example, the transceiver 197 receives measurement data from the blood pressure measurement apparatus 20.

### Other Embodiments

In the first embodiment, the blood pressure data processing apparatus is provided separate from the blood pressure measurement apparatus. In another embodiment, at least one or all of the constituent elements of the blood pressure data processing apparatus may be provided in the blood pressure measurement apparatus.

The blood pressure measurement apparatus is not limited to blood pressure measurement apparatus using tonometry, and may be a blood pressure measurement apparatus of any type that can continuously measure blood pressure. For example, it is possible to use a blood pressure measurement apparatus that non-invasively measures a pulse transit time (PTT), which is the transit time of a pulse wave being transmitted through an artery, and estimates the blood pressure value (e.g. the systolic blood pressure) based on the measured pulse transit time. It is also possible to use a blood pressure measurement apparatus that optically measures a plethysmogram. Also, it is possible to use a blood pressure measurement apparatus that non-invasively measures blood pressure using an ultrasonic wave.

The blood pressure measurement apparatus 20 is not limited to a wearable apparatus, and may be a stationary apparatus that performs blood pressure measurement in a state in which the upper arm of the measurement subject is placed on a fixing platform. A wearable blood pressure measurement apparatus does not restrict the measurement subject from moving, but the sensor unit 22 is likely to be displaced from a position that is suitable for measurement.

The peak detection interval setting unit 13 may use acceleration data to set peak detection intervals to the time-series data 11. For example, processing may be performed to detect body movement of the measurement subject based on acceleration data, and the peak detection interval setting unit 13 may exclude intervals in which body movement is detected, from peak detection intervals.

The present invention is not limited to the above-described embodiments as they are, and can be realized with modifications to the constituent elements without departing from the spirit thereof in the implementation stage. Also, various aspects of the invention can be formed through suitable combinations of the plurality of constituent elements disclosed in the above-described embodiments. For example, several constituent elements may be removed from all of the constituent elements shown in the embodiments. Furthermore, the constituent elements of different embodiments may be combined as appropriate.

At least one or all of the above-described embodiments can be described as in the following supplementary notes as well, but there is no limitation to the following description.

### Supplementary Note 1

A blood pressure data processing apparatus comprising:

a processor; and

a memory that is coupled to the processor,

wherein the processor is configured to:

acquire time-series data regarding a blood pressure value;

set one or more peak detection intervals to the time-series data, and calculate a feature amount for each of the peak detection intervals based on one of a systolic blood pressure, a diastolic blood pressure, and a pulse pressure; and

specify at least one first peak from the feature amount of each peak detection interval.

### Supplementary Note 2

A blood pressure data processing method comprising:

acquiring time-series data regarding a blood pressure value, using at least one processor;

setting one or more peak detection intervals to the time-series data, and calculating a feature amount for each of the peak detection intervals based on one of a systolic blood pressure, a diastolic blood pressure, and a pulse pressure, using at least one processor; and

specifying at least one first peak from the feature amount of each peak detection interval, using at least one processor.

1-12. (canceled)

13. A blood pressure data processing apparatus comprising:
an acquisition unit configured to acquire time-series data regarding successive blood pressure values;
a calculation unit configured to set a plurality of peak detection intervals moved along a time axis, to the time-series data, and calculate, for each of the plurality of peak detection intervals, a feature amount that includes a maximum value that is based on one of a systolic blood pressure, a diastolic blood pressure, and a pulse pressure;
an extraction unit configured to extract a peak candidate from each of the plurality of peak detection intervals by applying a determination criterion to the feature amount calculated for each of the plurality of peak detection intervals, and
a specifying unit configured to specify a first peak based on no less than a predetermined number of peak candidates that are present at the same point in time as a point in time at which the maximum value of the peak candidates can be acquired.

14. The blood pressure data processing apparatus according to claim 13,
wherein the feature amount includes a difference between the maximum value in the peak detection interval and a minimum value of one of the systolic blood pressure, the diastolic blood pressure, and the pulse pressure at a point in time that precedes the maximum value in the peak detection interval.

15. The blood pressure data processing apparatus according to claim 13,
wherein the specifying unit narrows down the at least one first peak using another feature amount that is based on at least one of a waveform, time information, and frequency information regarding the time-series data.

16. The blood pressure data processing apparatus according to claim 15,
wherein the other feature amount includes a rise time, a fall time, an area, and a correlation coefficient of a blood pressure surge.

17. The blood pressure data processing apparatus according to claim 13, further comprising:
a display unit configured to display the at least one first peak together with the time-series data.

18. The blood pressure data processing apparatus according to claim 13, further comprising:
a search unit configured to detect at least one second peak by searching for a local maximum value of the time-series data at at least one of a time point that precedes a search range that includes the at least one first peak and a time point that is subsequent to the search range.

19. The blood pressure data processing apparatus according to claim 18, further comprising:
a display unit that displays the at least one first peak and the at least one second peak together with the time-series data; and
a display control unit configured to control the display unit so as to display the at least one first peak and the at least one second peak so as to be distinguishable from each other.

20. A blood pressure data processing method comprising:
acquiring time-series data regarding successive blood pressure values;
setting a plurality of peak detection intervals moved along a time axis, to the time-series data, and calculate, for each of the plurality of peak detection intervals, a feature amount that includes a maximum value that is based on one of a systolic blood pressure, a diastolic blood pressure, and a pulse pressure;
extracting a peak candidate from each of the plurality of peak detection intervals by applying a determination criterion to the feature amount calculated for each of the plurality of peak detection intervals, and
specifying a first peak based on no less than a predetermined number of peak candidates that are present at the same point in time as a point in time at which the maximum value of the peak candidates can be acquired.

21. A program for causing a computer to function as the blood pressure data processing apparatus according to claim 13.
